(19) United States
(12) Patent Application Publication
(10) Pub. No.: US 2010/0030312 A1
(43) Pub. Date: Feb. 4, 2010

(54) METHOD AND APPARATUS FOR LEAD LENGTH DETERMINATION

(76) Inventor: Xiaonan Shen, Shoreview, MN (US)

Correspondence Address: MEDTRONIC, INC., 710 MEDTRONIC PARKWAY NE, MINNEAPOLIS, MN, 55432-9924, US

(21) Appl. No.: 12/183,592

(22) Filed: Jul. 31, 2008

(51) Int. Cl. A61N 1/00; A61B 5/05

(52) U.S. Cl. 607/122; 600/424

(57) ABSTRACT

A method and apparatus for positioning a lead within an anatomy is disclosed. The apparatus can be tracked relative to the anatomy to determine the position of selected portions of the anatomy. The positions of the portions of the anatomy can be used in determining an appropriate lead length to be positioned within the anatomy. Additionally, a determination of a lead length can be based upon statistical or acquired data of a selected population, according to an appropriate method.

### FIELD

The present disclosure relates to positioning an implantable device including a lead within an anatomy of a patient, and particularly to positioning an appropriate length of lead within a selected portion of the anatomy of the patient.

### BACKGROUND

This section provides background information related to the present disclosure which is not necessarily prior art.

In an anatomy of a patient, a medical device can be implanted. An implantable medical device (IMD) can include various devices, for example pacemakers, brain stimulation or neurostimulation devices, cardiac defibrillators, and other appropriate devices. The IMD's generally include at least two main portions, a case and leads extending from the case. The case can include a drive system, a power source or battery, various electronics, and other appropriate systems. The leads can interconnect with the case and include a lead tip or tip electrode that is positioned in an area within the anatomy to provide a therapy to a particular location in the anatomy.

A pacemaker IMD can be implanted within a chest wall or other appropriate location within the anatomy and leads can be positioned within the heart of the patient. The leads can carry an electrical stimulation from the case to the lead tip to provide an appropriate therapy. Leads may also extend from a case, according to various embodiments, to various neurological regions, including the brain and spinal cord. The leads can be positioned at appropriate locations to provide a therapy to the specific locations in the brain or spinal column as selected.

The leads include various portions, such as a conductor, casing, or sheath and other appropriate portions. The leads can be positioned using various systems, such as fluoroscopy. The lead can also be selected to extend a selected length from the case to the lead tip.

### SUMMARY

This section provides a general summary of the disclosure, and is not a comprehensive disclosure of its full scope or all of its features.

When a lead is positioned within the patient, the length of lead, between the case and the tip, can be selected according to various procedures. The length of the lead, generally the length of the conductor and other portions extending from the case to the tip, can be selected for movement of the patient and for movement of the specific anatomical portions of the patient. For example, when the case and the lead of the IMD are implanted, the patient may be lying supine on an operating room table. The patient, after the operation, may then be moved to a different orientation which can cause movement of the organs into which the lead was implanted. The amount of movement of the organs after implantation, such as when the patient moves from an implanted position to an activity position, can be accounted for by allowing an appropriate amount of slack in the lead.

The slack in the lead can include an additional amount of lead length that is provided, but not specifically necessary, to position the lead between the lead tip and the case during the implantation procedure. The amount of slack, however, can ensure that the lead tip remains at the implanted location selected by a user, such as a surgeon, during an operative procedure. Lead slack can be used to ensure long term fixation of the lead tip at the selected implant location.

Various tests or prior procedures can be used to determine an appropriate amount of lead slack and may depend upon average or general sizes of a patient anatomy. For example, an amount of lead slack when positioning a lead tip within an apex of the heart can be calculated based upon a statistical average of a population. Obtaining position measurements of various portions of the heart can be used to determine an appropriate amount of lead length to be provided within the heart. Tracking a stylet, catheter, or other appropriate lead delivery portion can be used to ensure that the appropriate or selected amount of lead length is provided. Accordingly, a tracking system and/or imaging system can be used to ensure an appropriate or selected amount of lead length is provided during an implantation procedure.

According to various embodiments, a lead system operable to be positioned in a selected volume is disclosed. The lead system can include a distal tip of a lead body operable to be moved within the volume and a tracking device moveable relative to the distal tip and substantially along an axis of the lead body. The system can further include a localizing system operable to track a position of the tracking device within the volume and a processor operable to determine a first location and a second location of the tracking device within the volume to determine a first dimension by tracking the tracking device. The distal tip is operable to be fixed within the volume and the tracking device is operable to be tracked relative to the fixed distal tip.

According to various embodiments, a method of positioning a lead system in a selected volume is disclosed. The method can include determining a dimension, determining a length of a lead based upon the determined dimension, and positioning the lead and a second member within the selected volume. The method can further include withdrawing the second member from the volume and measuring the movement of the second member to confirm that the determined length of the lead is within the volume. The measured length of the lead can also be implanted.

According to various embodiments, a method of positioning a lead system in a selected volume of a heart of a patient is disclosed. The method can include determining a general relationship between a dimension within the heart and a length of lead positioned within the heart to achieve a selected result, including: selecting a plurality of patients, selecting a location of a position of a lead tip, determining a first dimension in each of the plurality of hearts relative to the selected location of the lead tip, determining an appropriate length of the lead in each heart of the plurality of patients relative to the selected location of the lead tip, and determining a relationship between the determined first dimension and the determined appropriate length. A first dimension in the patient can be determined and determining a first length of lead to be positioned in the patient can be based upon the determined relationship using the determined first dimension in the patient. A lead assembly can be positioned within the heart of the patient and measuring a length of lead within the heart of the patient as determined with the relationship can be performed. The measured length of the lead can also be implanted.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an exemplary view of a trackable electrode within a heart;

FIG. 2 is an exemplary graphical illustration of the relationship of an anatomical dimension and lead length;

FIGS. 3A-3D illustrate a cross-sectional detailed view of a lead in various orientations relative to an anatomy, according to various embodiments;

FIGS. 4A-4D illustrate a detailed cross-sectional view of a lead positioned relative to an anatomy portion, according to various embodiments; and

FIGS. 5A-5D illustrate an exemplary method of positioning a lead in a heart.

Corresponding reference numerals indicate corresponding parts throughout the several views of the drawings.

### DETAILED DESCRIPTION

Example embodiments will now be described more fully with reference to the accompanying drawings.

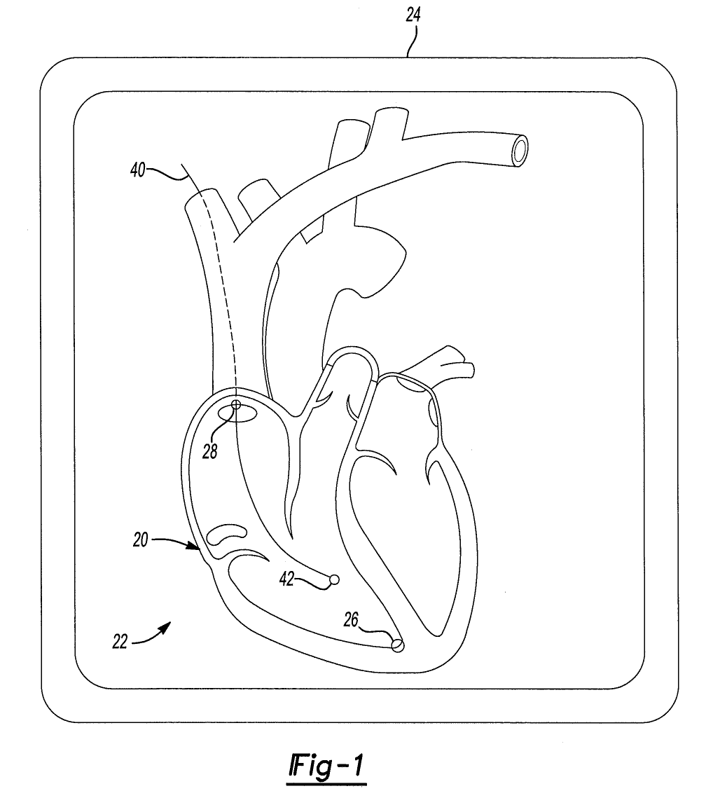

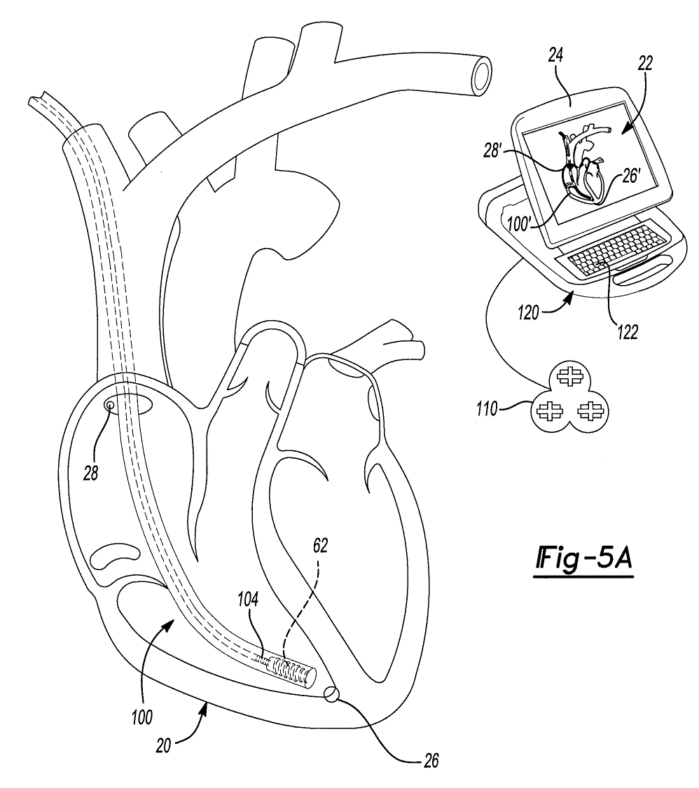

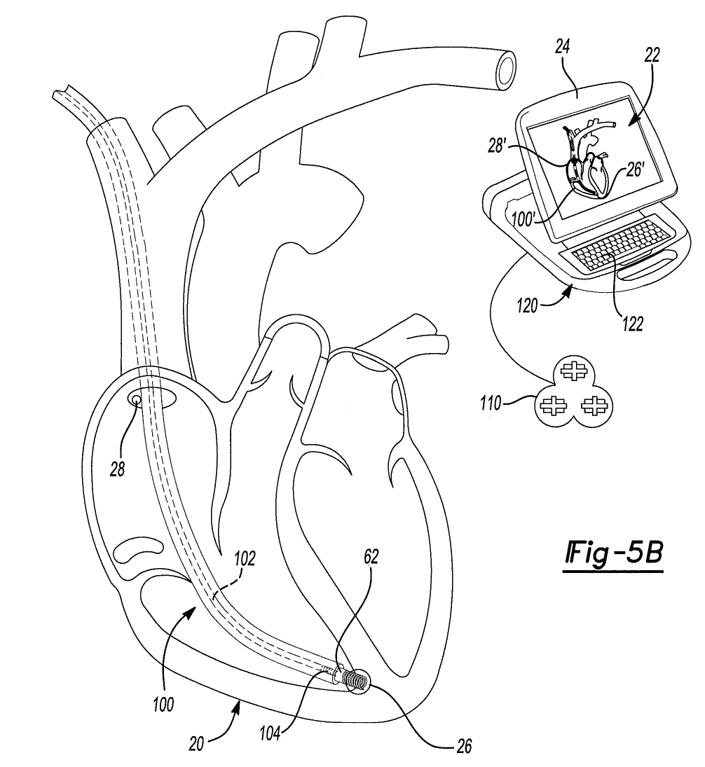

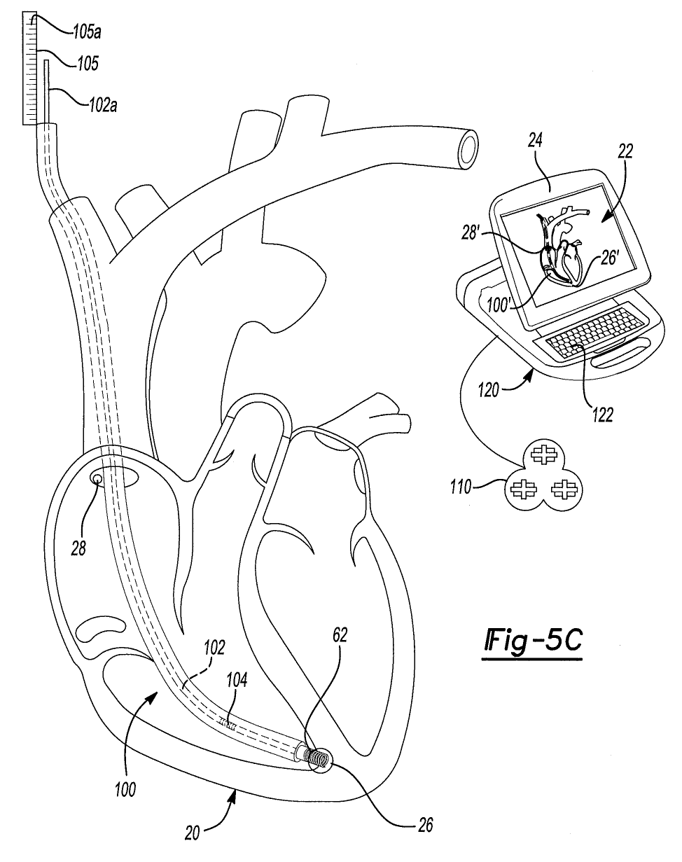

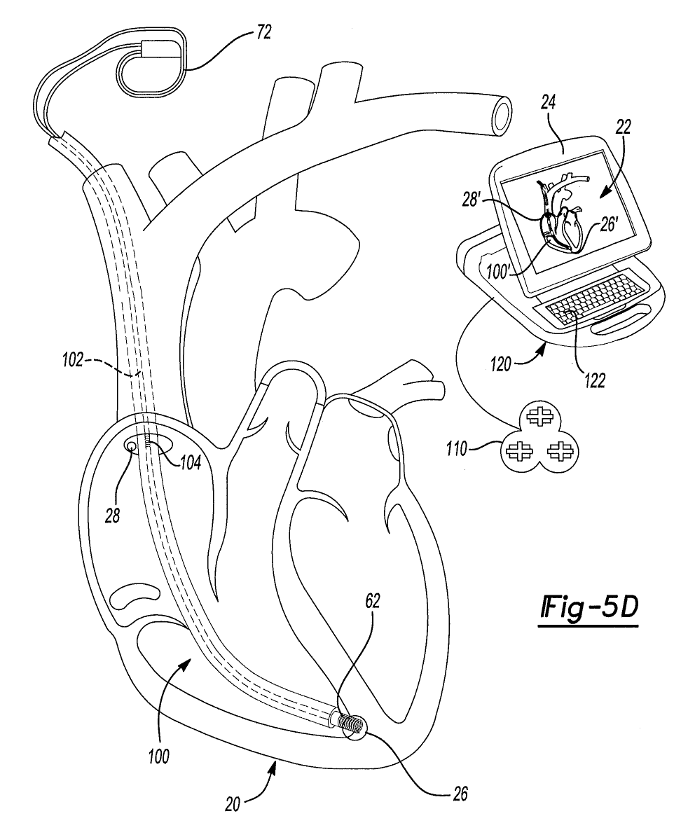

With reference to FIG. 1, a heart 20 of a patient can be selected to include a lead implanted during a selected procedure. The heart 20 can be illustrated with image data 22 on a display 24 (See FIGS. 5A-5D). The display 24 can display two dimensional or three dimensional image data of the heart 20. The heart 20 can include a right ventricle (RV) apex 26 and a superior vena cava/right atrium (SVC/RA) junction 28. It will be understood that the image data 22 can be acquired with any appropriate modality, such as a three dimensional modality, so a distance or other appropriate geometry between the RV apex 26 and the SVC/RA junction 28 can be measured. As discussed further herein, various additional procedures can also be used to determine the position of or dimension between the RV apex 26 and the SVC/RA junction 28.

The determination of the position of the RV apex 26 and the SVC/RA 28, however, can be used to determine a dimension between the RV 26 and the SVC/RA 28. Accordingly, measurements can be made using appropriate image data, such as MRI or CT image data, or with other appropriate mechanisms. For example, direct anatomical measurements may be made of a patient or a population of patients.

According to various embodiments, a lead 40, exemplary illustrated relative to the heart 20, can be moved through the heart 20 of a patient to contact or be positioned relative to both the RV apex 26 and the SVC/RA 28 to directly measure a dimension between the RV apex 26 and the SVC/RA 28. The lead 40 can include a tracked portion, including a tracking device 42, that can be tracked in space to determine a spacial coordinate of the RV apex 26 and the SVC/RA 28 junction. The tracking device 42 can be any appropriate tracking device, such as one used with an electromagnetic tracking system, an electrical potential tracking system, an acoustic tracking system, an ultrasonic tracking system, or any other appropriate tracking system. Appropriate tracking systems can include those disclosed in U.S. patent application Ser. No. 12/117,537, filed on May 8, 2008 and U.S. patent application Ser. No. 10/619,216, filed on Jul. 14, 2003, both incorporated herein by reference. It will be understood that the movement of the heart 20, or other anatomical features, may be accounted for or averages when determining the anatomical dimensions.

The dimension or distance between the RV apex 26 and the SVC/RA junction 28 can be used to determine an appropriate amount of lead length to be positioned within the heart 20 during an implantation procedure. The amount of lead length positioned within the heart 20 can be based upon a population or statistical average of the distance between the RV apex 26 and the SVC/RA junction 28. For example, a plurality or population of patients can be measured at various positions, such as prone, lying supine, standing, and the like. The measurements can be obtained using appropriate image data, such as three dimensional image data, or with tracking the tracked catheter 40. After an appropriate population has been selected, the measurements can be obtained and the measurements can be used to determine the appropriate amount of lead length.

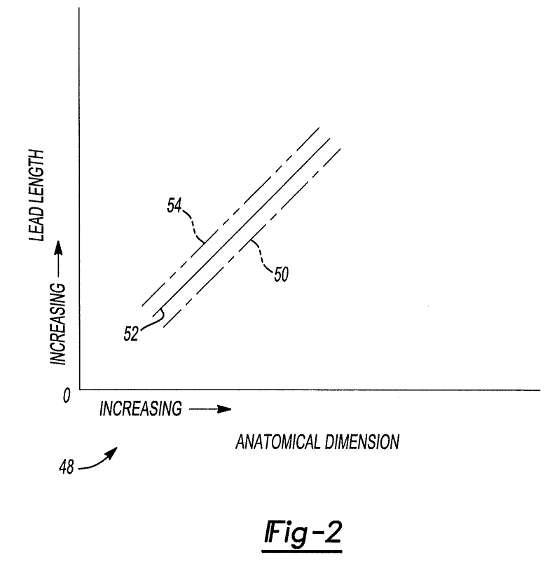

With additional reference to FIG. 2, an appropriate lead length to anatomical dimension relationship 48 can be determined. The relationship 48 can be any appropriate relationship, such as a substantially linear relationship as illustrated in FIG. 2. As illustrated in the graph in FIG. 2, an anatomical dimension can be plotted on an X-axis and an appropriate lead length can be plotted on a Y-axis. The anatomical dimension can be the dimension between the RV apex 26 and the SVC/RA junction 28 and can be measured in any appropriate units, such as millimeters. Any other selected dimension can also be measured. The lead length can be the appropriate lead length required or selected to be positioned within the heart 20 to ensure an appropriate amount of slack or tension of the lead within the heart 20. The tension can be between the tip of the lead and the case of the IMD 20.

The relationship 48 illustrated in FIG. 2 can be provided for viewing by a user or as a specific formula that can be calculated and illustrated for a user or calculated by a user. As discussed herein, the relationship 48 can be used to calculate and an appropriate range, such as a minimum length 50, an average or optimal length 52, and a maximum length 54 of lead. Regardless of the specific formula of the relationship 48, it can be used to determine an appropriate lead length based upon the anatomical dimension, such as the distance between the RV apex 26 and the SVC/RA junction 28.

The relationship 48 can be determined in any appropriate manner. For example, a population of successful implant recipients can have both an anatomical dimension and a lead length measured. The measurements of the successful population can be used to determine the relationship 48. In addition, various clinical or research studies can be used to determine the appropriate relationship. Regardless of the method used, the relationship 48 can be determined between the selected anatomical dimension and an appropriate lead length.

The anatomical dimension, however, can be any appropriate dimension and be based on any appropriate population. For example, the anatomical dimension can also be a dimension between the SVC/RA junction 28 and the coronary sinus ostium, a pulmonary outflow, or other selected location. The anatomical dimension, regardless of the specific dimension of the anatomy of the patient, can be based upon a population or selected plurality of patients. The measurements can be acquired as discussed above or can be acquired in any appropriate manner. Acquisition of data of the selected number of patients, however, can be used to determine, an amount of lead length to be implanted in the patient during a specific procedure. Accordingly, the length of lead can be determined prior to a specific procedure for a measured anatomical dimension. The lead length, therefore, is predetermined.

According to various embodiments a tracking device and/or a measuring device can be provided with a lead assembly. As discussed herein, either or both of the tracking device and the measuring device can be used when measuring or determining the length of lead being implanted. It will be understood, that the tracking device or the measuring device can be used alone or together to measure the lead length in the patient or organ, such as the heart 20.

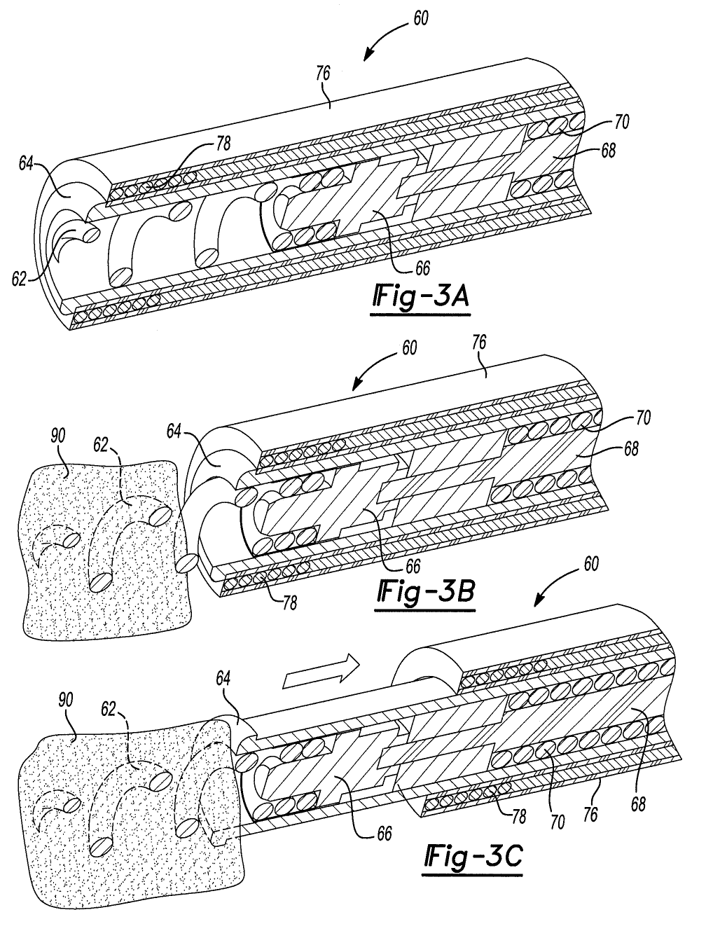

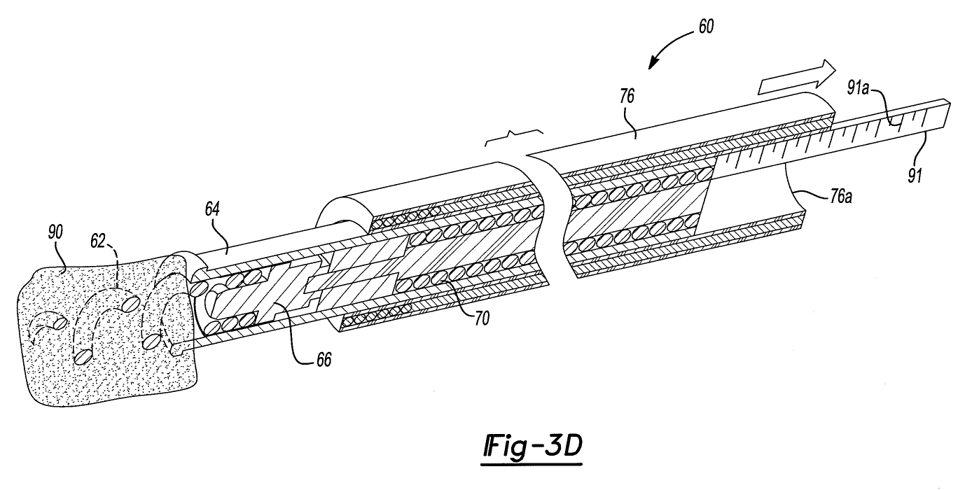

According to various embodiments, as illustrated in FIGS. 3A-3D, a distal portion of a lead assembly 60 is illustrated. The lead assembly 60 can include any appropriate lead assembly, such as the Quattro lead assembly sold by Medtronic, Inc., having a place of business in Minneapolis, Minn. The lead assembly 60 can generally include a distal tip or tip electrode 62 that can define a helix. The tip electrode 62 can be retractable or extendable relative to a sheath 64. It will be understood, however, that the tip electrode 62 can also be fixed relative to the sheath 64 in a position extended from the sheath 62.

According to various embodiments, however, the tip electrode 62 can extend from the sheath 64 by rotation or other movement of a connecting portion 66. The connecting portion 66 can be driven or moved with a stylet 68 that extends along the length of the sheath 64 at least during positioning of the lead assembly 60. In addition, a conductor 70 can be provided substantially the length of the lead assembly 60 to transmit a current or signal from an IMD case 72 (FIG. 5D) for pacing, defibrillation, or the like. It will be understood that the lead assembly 60 can include other generally known portions and can be implanted in any appropriate manner, as is generally known in the art.

The lead assembly 60, however, can further include a positioning catheter 76. The positioning catheter 76 can surround the sheath 64 of the lead assembly 60. The positioning catheter 76 can be formed of any appropriate material, such as appropriate polymers. The positioning catheter 76 can further include one or more tracking devices 78. The tracking device 78 can be used with any appropriate navigation system, such as an electromagnetic or electrical potential navigation system. The tracking device 78 can include a coil, electrode, ultrasound transducer, etc. Therefore, the tracking device 78 can be used to track any appropriate portion of the lead assembly 60, such as a distal portion of the lead assembly 60.

For example, when the tracking device 78 of the positioning catheter 76 is positioned substantially near an end or distal end of the sheath 64, the tracking device 78 can be used to track the tip of the lead assembly 60. Briefly, and as discussed herein, the lead assembly 60 can be moved through a selected portion of the anatomy, such as the heart 20, to position the lead assembly 60 near a tissue portion 90, as illustrated in FIG. 3B. When the lead assembly 60 is positioned near the tissue 90, the tip electrode 62 can be extended and positioned within the tissue 90, such as with the stylet 68. As discussed above, the tip electrode 62 can be extended with the stylet 68 or can be moved into the tissue 90 because it is permanently extended from the sheath 64. Regardless of the insertion method, the tip electrode 62 can be positioned within the tissue 90.

As illustrated in FIG. 3C, the positioning catheter 76 can be withdrawn along the length of the sheath 64 of the lead assembly 60. As the positioning catheter 76 is moved along the length of the lead sheath 64, the tracking device 78 can be tracked. This allows the tracking device 78 to be tracked as it moves from the position of the implantation of the tip electrode 62 to any other appropriate position. As illustrated, particularly in FIG. 3B, the tracking device 78 can be positioned substantially adjacent or near the location of the implantation of the tip electrode 62. This position can be any appropriate position, such as the RV apex 26. As the positioning catheter 76 is moved along the length of the sheath 64, the position of the tracking device 78 can be tracked as the positioning catheter 76 is moved. Accordingly, a selected length of lead can be calculated due to a displacement amount of the tracking device 78 as the positioning catheter is moved relative to the sheath 64 of the lead assembly 60 from a first location to a second location along an axis of the sheath 64.

As illustrated in FIG. 3D, as the positioning catheter 76 is withdrawn over the sheath 64, a distal portion of the positioning catheter 76a can be measured with a measuring device or ruler 91. The measuring device 91 can include demarcations 91a. The position of the SVC/RA juncture 28 and the RV apex 26 can be determined with the tracking device 78 of the lead assembly 60. As the tip electrode 62 is passed through the heart 20. The anatomical dimension between these two points can then be used with the relationship 48 to determine the length of lead to be positioned within the heart 20. The measuring device 91 can be used to measure the length of lead being uncovered and left within the heart 20 as the positioning catheter 76 is withdrawn. In other words, if the anatomical dimension is used to calculate the lead length, the measuring device 91 can be used to measure the actual length of the lead in the heart 20 as the catheter 76 is withdrawn. Accordingly, the measuring device 91 can assist in determining the amount of lead or length of lead being left within the heart 20.

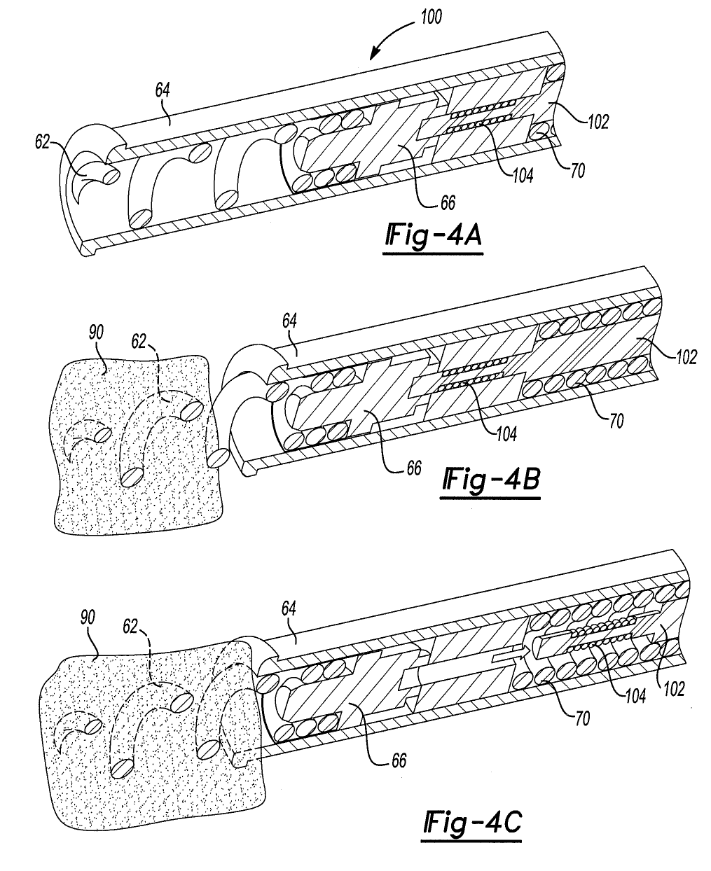

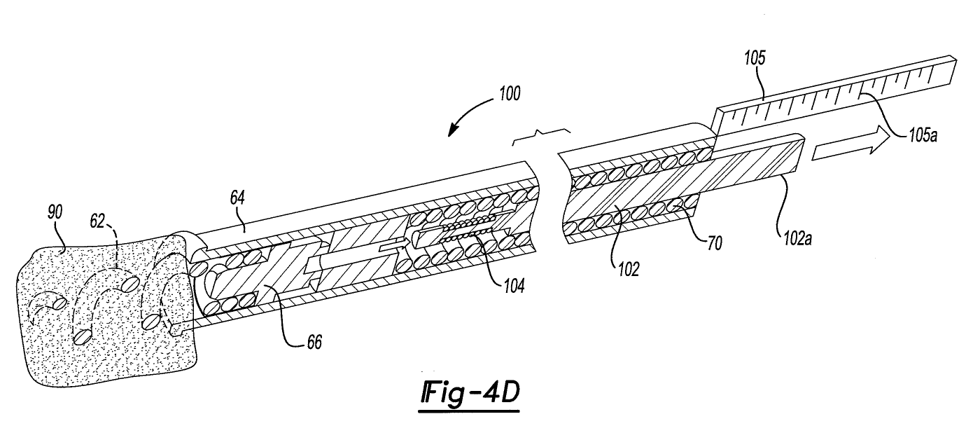

According to various embodiments, as illustrated in FIGS. 4A-4D, a lead assembly 100 is illustrated. The lead assembly 100 can include portions that are substantially similar to the lead assembly 60 which are numbered with substantially identical numerals and mentioned only briefly here. Generally the lead assembly 100 can include the sheath 64 and the tip electrode 62. Again, one skilled in the art will understand, that the tip electrode 62 can be a retractable electrode or a fixed electrode relative to the sheath 64. The lead assembly 100 can further include the conductor 70 and the connecting portion 66 that interconnects the conductor 70 with the tip electrode 62.

The lead assembly 100 can also include a stylet 102 similar to the stylet 68 for positioning the tip electrode 62 relative to the tissue 90. The stylet 102, however, can further include or incorporate a tracking device 104. The tracking device 104 can be used with any appropriate tracking system, such as an electromagnetic, electropotential, or any other appropriate tracking system used with the tracking device 78 discussed above.

As illustrated in FIG. 4B, the lead assembly 100 can be positioned relative to the tissue 90, similar to the lead assembly 60. Again, the lead assembly 100 can be interconnected with the case 72 of the IMD for appropriate purposes, such as pacing or defibrillation. When the lead assembly 100 is positioned relative to the tissue 90, the stylet 102 can be positioned at a known location relative to the tip electrode 62. For example, the tracking device 104 of the stylet 102 can be positioned substantially in connection with the connector 66, which includes a known location relative to the tip electrode 62. Accordingly, a tracked location of the tracking device 104 can be known substantially precisely for determining a position of the tip electrode 62 relative to the anatomy or at a physical location.

The stylet 102 can be withdrawn through the lead assembly 100. The stylet 102, including the tracking device 102 can be withdrawn through the conductor 70 and sheath 64. The tracking device 104 can be tracked as it moves relative to the implanted tip electrode 62. As the tracking device 104 moves with the stylet 102 through the sheath 64 of the electrode assembly 100, a position of the tracking device 104 can be determined. The tracking device on the stylet 102 can be tracked or navigated as it moves to any appropriate portion of the anatomy, such as the SVC/RA junction 28 of the heart 20.

When the tracking device 104 is tracked as the lead assembly 100 is moved relative to the heart 20 its position can be determined and illustrated relative to the image data 22. The image data 22 can be acquired at any appropriate time, such as preoperatively or intraoperatively. Additionally, the image data 22 can be registered to the patient space as is generally understood by one skilled in the art. Therefore, as the tracking device 104 is moved relative to the heart 20, a position of the tracking device 104 and other appropriate portions of the lead assembly 104 can be determined. In addition, icons can be displayed on the display device 24 relative to the image data 22 to illustrate the positions of the various portions of the lead assembly 100.

When the lead assembly 100 is moved within the heart 20, the tracking device 104 can be used to identify various anatomical landmarks, such as the position of the SVC/RA junction 28. The determination of the anatomical landmarks can be with any appropriate method, such as pulsitive pressures, surgeon knowledge, imaging (e.g. fluoroscopy), or any other appropriate mechanism. The position of the SVC/RA junction 28 can be used in conjunction with other appropriate landmarks, such as the RV apex 26 to determine the anatomical dimension. Therefore, by tracking the tracking device 104 positions of appropriate anatomical landmarks can be determined.

With reference to FIG. 4D, the lead assembly 100 can also include a measuring device 105. The measuring device 105 can include demarcations 105a for measuring a distance that the stylet 102 has moved out of the sheath 64. The withdrawal length of the stylet 102 can be used to determine the length of lead left within the heart 20. As discussed above, as the tracking device 104 is tracked relative to the heart 20, various landmarks can be determined. Also, as further discussed above, the relationship of the dimension of various anatomical portions can be used to determine the length of lead to be positioned within the heart 20. Accordingly, as the stylet 102 is withdrawn, the measuring device 105 can be used to ensure that an appropriate determined length of lead is positioned within the heart 20. Similar to the measuring device 91, discussed in relationship to the lead assembly 60, the measuring device 105 can be used to measure the length of the stylet 102 withdrawn from the lead assembly 100 for determining the length of lead positioned within the heart 20.

As discussed above, lead assemblies, according to various embodiments, can include tracking devices that are used to track positions of at least a portion of the lead assemblies. As also discussed above, a lead length can be determined based upon an anatomical dimension measured in a patient. According to various embodiments, for example as illustrated in FIGS. 5A-5D, an exemplary method of positioning a lead within the heart 20 and determining or verifying that a selected lead length is left in the heart 20 is illustrated and described herein.

A tracking system can include the tracking devices 42, 78, and 104 and associated localizing systems. Localizing systems can include an electromagnetic localizer 110. The electromagnetic localizer 110 can include one or more coils to generate a field or sense a field from the heart 20, or other appropriate portion of the patient. The sensed location with the field can be used to determine the location of the tracking device 42, 78, and 104. It will be understood that other localizing systems can be provided, such as bioimpedance localizing systems, ultrasound localizing systems, etc. In addition, it will be understood, that the lead assemblies 60, 100 or any appropriate lead assemblies, may have more than one tracking device associated therewith. When more than one tracking device is present on a single lead assembly more tracked points can be determined or tracked for that lead assembly.

The patient, including the heart 20, can define a patient space. The patient space can be registered to image data acquired of the patient at any appropriate time. The image data can be any appropriate image data, such as fluoroscopic image data, MRI image data, etc. The image data can be 2D, 3D, or 4D. The image data can be registered to the patient space in appropriate known methods, such as fiducial, landmark, or point matching.

Once the image data is registered to the patient space a representation, for example an icon, can be superimposed on the image data 22 on the display 24. The icons can represent landmarks or tracking devices, as discussed herein. A user can then view a position of an instrument or other portion relative to the image data on the display 24 as the tracked portion is positioned relative to the patient, such as the heart 20, in patient space.

With initial reference to FIG. 5A, the stylet 102 including the tracking device 104 can be used to help insert the tip electrode 62 with an appropriate localizer 110. As discussed above, the tip electrode 62 can be positioned in any appropriate portion of the anatomy, such as the heart 20. As a further example, the distal tip electrode 62 can be positioned within or at the RV apex 26.

A processor system 120 including the display device 24 can display the image data 22 of the heart 20. The processor system 120 can include appropriate input devices, such as a keyboard 122. The display device 24 can further display icons, such as a lead assembly icon 100′; an icon representing the determined position of the RV apex 26′, and an icon representing the position of the SVC/RA juncture 28′. The tracked position of the tracking device 104 can be used to determine the position of the RV apex 26 and the SVC/RA juncture 28 and they can be displayed as icons on the display device 24. It will be understood, however, that an icon need not be displayed on a display device 24.

The image data 22 can be any appropriate type of image data. For example, the image data 22 can include three dimensional image data that can include information regarding geometrical or distances between different points in the anatomy. Alternatively, a fluoroscopic image or other X-ray image can be used, which is two dimensional. The image data can be registered to the anatomy or patient space, as is understood by one skilled in the art. The tracked position of the tracking device 104 can then be used to determine the position of the RV apex 26 when the lead assembly 100 is positioned within the heart 20. For example, as illustrated in FIG. 5B, when the distal tip electrode 62 is positioned within the RV apex 26 of the heart 20, the tracking device 104 can be used to identify the position of the RV apex 26. Further, as discussed above, the RV apex icon 26′ can be displayed on the display device and superimposed on the image data 22.

As discussed above, as the lead assembly 100 is moved relative to the heart 20, the position of the SVC/RA junction 28 can be determined and its position can be measured. Various mechanisms, such as pulsative pressure, and other techniques can be used to determine the position of the SVC/RA junction 28. Pressure sensors can be provided with the lead assembly 100 to measure pulsative pressure. In addition, electrograms or anatomical measurements can be used to assist in determining the anatomical location of various landmarks. For example, the distal tip electrode 62 can measure voltages that can be used to determine when the electrode is at or near the SVC/RS juncture 28. The position of the landmarks or anatomical points, however, can be displayed as the SVC/RA icon 28′ on the display device 24. Again, the image data 22 can include the SVC/RA icon 28′ and the RA apex icon 26′ superimposed thereon.

With continuing reference to FIG. 5B, the distal tip electrode 62 can be positioned in the RV apex 26 according to any appropriate method, including those understood by one skilled in the art. For example, as briefly discussed above, the stylet 102 can extend to the tip electrode 62 and further be used to move the tip electrode 62 into the tissue of the heart at the RV apex 26. Once the tip electrode 62 is appropriately positioned within the RV apex 26, the stylet 102 can be positioned at a known position relative to the tip electrode 62. The tracking device 104 at the known position relative to the tip electrode 62 can be used to determine a location at the RV apex 26 due to the known location of the tip electrode 62. The determination of the position of the RV apex 26 can then be made substantially precisely.

With reference to FIG. 5C, once the tip electrode 62 is positioned within the RV apex 26, the stylet 102 can be selectively withdrawn from a position at or near the tip electrode 62 to any appropriate position within the lead assembly 100. As discussed above, the tracking device 104 can be tracked to determine its position relative to the heart 20 or any portion of the anatomy or the lead assembly 100. Accordingly, a length of the lead between the determined position of the RV apex 26 and any other appropriate portion of the anatomy can be determined by tracking the tracking device 104 within the heart 20. The position of the tracking device 104 can be tracked as it is moved towards the SVC/RA junction 28 of the heart 20. Further, as the tracking device 104 is tracked, its position relative to the SVC/RA junction 28 can be tracked and displayed on the display device 24.

As the stylet 102 continues to be withdrawn from the lead assembly 100 and away from the distal tip electrode 62, the length of lead is effectively being let out. As discussed above, the anatomical dimension between the RV apex 26 and the SVC/RA junction 28 can be used to determine length of lead to be positioned within the heart 20. Accordingly, after having measured the anatomical dimension between the SVC/RA junction 28 and the RV apex 26 within the heart 20 of the specific patient, an appropriate length of lead can be calculated based upon the relationship 48. As the stylet 102 is withdrawn from or away from the distal tip electrode 62, the length or amount of withdrawal of the stylet 102 can be used to determine the length of lead left within the heart 20. In other words, the movement of the stylet 102 relative to the tip electrode 62 can be used to determine the length of the lead sheath 64, conductor 70, and other portions of the lead assembly or body 100 within the heart 20.

Also, as illustrated in FIG. 5C, and briefly discussed above, the measuring device 105 including the demarcations 105a can be used to measure the length of the stylet withdrawn from the sheath 64 of the lead assembly 100. Based upon the determined lead length to be positioned within the heart 20, the stylet 102 can be withdrawn the appropriate amount. Once the stylet has been withdrawn the appropriate amount, as measured with the measuring device 105, with the tracking system, or any other appropriate mechanism, the stylet and the sheath 64 can be fixed relative to the heart 20. As discussed further herein, the case 72 can then be positioned or the lead assembly 100 can be interconnected with the case 72. This can assist in substantially fixing the lead assembly 100 relative to the heart 20.

As illustrated in FIG. 5D, once a portion of the stylet, such as a proximal portion 102a of the stylet 102 including the tracking device 104 reaches the SVC/RA junction 28 or when an appropriate distance has been traversed with the stylet 102, the length of lead can be determined that remains within the heart 20. For example, the anatomical dimension between the SVC/RA junction 28 and the RV apex 26 in the heart 20 can be used to determine that ten millimeters of lead length is to be positioned within the heart 20. Thus, the stylet 102 can be withdrawn ten millimeters. The length of withdrawal of the stylet can be determined by using the tracking device 104 to track when the stylet 102 has moved the appropriate length. Also, the measuring device 105 can be used to measure the distance that the stylet 102 has been withdrawn.

Once the stylet 102 has been moved the appropriate length, the lead assembly 100 can then be positioned within the anatomy and associated with the case 72 of the IMD. The lead assembly 100 can further be fixed relative to the heart 20 using appropriate fixation techniques. Accordingly, an appropriate amount of lead length can be positioned within the heart 20 to ensure an appropriate amount of lead slack. As discussed above, an appropriate amount of lead slack can be used to assist in ensuring an appropriate fixation of the distal tip electrode 26 relative to the heart 20.

It will be further understood that any appropriate anatomical dimension or lead length can be determined which can also vary with an individual patient or anatomical positioning of the distal tip electrode 62. As discussed above, the anatomical dimension and the lead length dimension can vary if the distal tip electrode 62 is positioned within a left ventricle of the heart 20 the pulmonary outflow, various neurological positions, or the like. Accordingly, appropriate information can be acquired for a population to be used with any appropriate implantation procedure.

In addition, the above disclosure is exemplary. According to various embodiments, electrodes on a lead assembly can be used with a navigation or tracking system, such as an electro-potential (EP) tracking system. Each electrode of a lead, such as an RV apex electrode and a SVC electrode could be tracked with the EP tracking system. Thus, according to various embodiments, a lead with more than one electrode can be used to provide more than one anatomical location or position at once with the multiple electrodes.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A lead system operable to be positioned in a selected volume, comprising:
a distal tip of a lead body operable to be moved within the volume;
a tracking device moveable relative to the distal tip and substantially along an axis of the lead body;
a localizing system operable to track a position of the tracking device within the volume;
a processor operable to determine a first location and a second location of the tracking device within the volume to determine a first dimension by tracking the tracking device;
wherein the distal tip is operable to be fixed within the volume and the tracking device is operable to be tracked relative to the fixed distal tip.

2. The lead system of claim 1, further comprising:
a display device operable to display image data of the volume;
wherein the processor is operable to display a first icon representing the first location relative to the image data and a second icon representing the second location relative to the image data.

3. The lead system of claim 1, further comprising:
a memory system including a relationship of a dimension between the first location and the second location and a length of the lead;
wherein the processor is operable to access the memory system based upon the determined first location and the second location and output a determined length of the lead.

4. The lead system of claim 1,
wherein the lead body includes a sheath extending a length from the distal tip;
wherein the tracking device is operable to move a distance along the length.

5. The lead system of claim 1,
wherein the tracking device is operable with the location system to determine a spatial location of a first volume location and a second volume location;
wherein a dimension between the first volume location and the second volume location is operable to be determined;
a measuring device operable to measure the length of the lead body with the volume related to the dimension.

6. The lead system of claim 5, wherein the lead body includes a sheath;
wherein the measuring device is fixedly positioned relative to a proximal end of the sheath.

7. The lead system of claim 1 further comprising:
a measuring device fixedly placed at a position on the lead body; and
a positioning member moveable with the distal tip and moveable relative to the distal tip;
wherein the positioning member is operable to be moved relative to the distal tip and measured with the measuring device.

8. The lead system of claim 7, further comprising:
a case assembly operable to be positioned within the volume and connected to a proximal end of the lead body;
wherein the case is operable to contain a pacing system operable to be programmed to deliver a signal to the distal tip.

9. The lead system of claim 1, further comprising:
a display device operable to display a tracked position of the tracking device;
wherein an icon representing a tracked position of the tracking device is operable to be superimposed on image data of the volume.

10. A method of positioning a lead system in a selected volume, comprising:
determining a dimension;
determining a length of a lead based upon the determined dimension;
positioning the lead and a second member within the selected volume;
withdrawing the second member from the volume;
measuring the movement of the second member to confirm that the determined length of the lead is within the volume; and
implanting the measured length of the lead.

11. The method of claim 10, wherein determining a length of the lead based upon the determined dimension, includes:
measuring a plurality of dimensions in a population;
determining an appropriate length of the lead based upon the plurality of measured dimensions; and
determining a relationship between that measured plurality of dimensions and the determined appropriate length of the lead.

12. The method of claim 10, further comprising:
moving a lead tip within the volume;
tracking a tracking device positioned relative to the lead tip as the lead tip is moved within the volume; and
determining a first location in the volume and a second location in the volume with the tracking device, wherein determining a dimension includes determining the dimension between the first location in the volume and the second location in the volume.

13. The method of claim 12, wherein measuring the movement of the second member includes:
tracking a tracking device associated with the second member as the second member moves form a first position to a second position; and
determining the distance between the first position and the second position.

14. The method of claim 12, wherein measuring the movement of the second member includes:
providing a measuring device fixed relative to the lead; and
measuring the length of the second member removed from the lead.

15. The method of claim 10, wherein implanting the measured length of the lead includes fixing a portion of the lead to a case member and positioning the case member within the volume.

16. A method of positioning a lead system in a selected volume of a heart of a patient, comprising:
determining a general relationship between a dimension within the heart and a length of lead positioned within the heart to achieve a selected result, including:
selecting a plurality of patients;
selecting a location of a position of a lead tip;
determining a first dimension in each of the plurality of hearts relative to the selected location of the lead tip;
determining an appropriate length of the lead in each heart of the plurality of patients relative to the selected location of the lead tip;
determining a relationship between the determined first dimension and the determined appropriate length;
determining the first dimension in the patient;
determining a first length of lead to be positioned in the patient based upon the determined relationship using the determined first dimension in the patient;
positioning a lead assembly within the heart of the patient;
measuring a length of lead within the heart of the patient as determined with the relationship; and
implanting the measured length of the lead.

17. The method of claim 16, wherein positioning a lead assembly includes:
providing a positioning device with the lead assembly.

18. The method of claim 17, wherein measuring a length of lead includes:
tracking a tracking device associated with the positioning device as the positioning device moves form a first position to a second position; and
determining the distance between the first position and the second position.

19. The method of claim 17, wherein measuring the movement of the second member includes:
providing a measuring device fixed relative to the lead assembly; and
measuring the length of the positioning device removed from the lead.

20. The method of claim 16, further comprising:
implanting a therapy delivery device; and
fixing the lead to the therapy delivery device.
